(19) United States
(12) Patent Application Publication
(10) Pub. No.: US 2026/0015679 A1
(43) Pub. Date: Jan. 15, 2026

(54) METHODS AND SYSTEMS FOR DETECTION AND IDENTIFICATION OF PATHOGENS AND ANTIBIOTIC RESISTANCE GENES

(71) Applicant: HAYSTACKANALYTICS PRIVATE LIMITED, Mumbai (IN)

(72) Inventors: Anirvan CHATTERJEE, Mumbai (IN); Amrutraj ZADE, Mumbai (IN); Priyanku KONAR, Mumbai (IN); Sanchi SHAH, Mumbai (IN); Sanjana KURUWA, Mumbai (IN); Mahua DASGUPTA KAPOOR, Mumbai (IN)

(21) Appl. No.: 18/995,223

(22) PCT Filed: Jul. 18, 2023
(86) PCT No.: PCT/IN2023/050698

§ 371 Date: Jan. 16, 2025

(30) Foreign Application Priority Data
Jul. 18, 2022 (IN) 202221041098

(51) Int. Cl. C12Q 1/689

(52) U.S. Cl. CPC C12Q 1/689; C12Q 2600/106; C12Q 2600/156

(57) ABSTRACT

Methods and systems for detection and identification of pathogens and antibiotic resistant genes are disclosed herein. The method is a high throughput, rapid comprehensive method for identification of pathogens in a sample or subject. The method, according to embodiments herein, is also capable of identifying genes responsible for Antimicrobial Resistance (AMR) in a pathogen. The said method provides a rapid, cost-effective and scalable solution for identifying pathogens.

### CROSS REFERENCE TO RELATED APPLICATIONS

This application is based on and derives the benefit of Indian Provisional Application IN202221041098, the contents of which are incorporated herein by reference.

### TECHNICAL FIELD

The present invention relates to methods and systems for detection and identification of pathogens and antibiotic resistance genes (ARGs). More specifically, the embodiments relate to rapid detection and identification of pathogens using sequence specific primers, performing genomic analysis and interpreting the analysis in context of relevant metadata.

### BACKGROUND

Rapid and accurate profiling of infection-causing pathogens remains a significant challenge in modern health care. There is the need for early and accurate identification of pathogens and their drug resistance profiles particularly in critical cases such as sepsis infections. Sepsis, a severe systemic inflammatory response to an infection, is also one of the most common causes of mortality for hospitalized patients worldwide. In low- and middle-income countries like India, the disease burden is high, with nearly 54% ICU patients having suspected or proven infection primarily caused by bacteria or fungi. In several cases infections are treated empirically. Delayed recognition of infections and inappropriate initial antibiotic therapy is associated with an increase in morbidity and mortality.

The current infection diagnostics techniques are mostly based on culture-based identification, antibody- or antigen-based assays and molecular-based approaches. For most samples, the turnaround time for culture-based identification techniques is usually around 2 to 4 days. However, for samples having low bacterial load, such as, bloodstream infection (BSI), a longer time is required. These tests suffer from limitations, such as, only microbes capable of growing under optimized culture conditions can be identified. Further these tests are laborious and time consuming as identification and antibiotic susceptibility testing (AST) for samples could take up to 3-25 days.

Timely diagnosis and efficient identification of an infectious agent are vital to providing early targeted antimicrobial intervention. It can substantially increase the survival rates, prevent ensuing complications, and decrease drug-related adverse events and eventually the cost burden. Rapid identification of clinically relevant antimicrobial resistance (AMR) encoding genes provides early optimization of suitable antibiotic therapy, thereby saving lives. ELISA-based hybridization, fluorescence-based Realtime detection, liquid or solid phase microarray detection, Sanger sequencing and MALDI-TOF MS are some of the molecular diagnostic methods that are used for diagnosis and identification of an infectious agent. However, these methods lack comprehensiveness and can only detect a few specific pathogens.

Polymerase chain reaction (PCR) based assays have been the gold standard in nucleic acid based microbial detection and diagnostics due to its ease of use, widespread instrument availability, and relatively low cost. However, PCR-based methods suffer from host DNA abundance and DNA fragments which are evolutionarily conserved between humans and bacteria. Also, some of them do not facilitate identification of antibiotic resistance genes or have limited panels. On the other hand, sequencing technologies offer highly sensitive and accurate method for detecting a wide range of pathogens and antibiotic resistance genes in a short period of time. However, the sensitivity of sequencing technologies is prone to be contaminated by non-pathogenic nucleic acid material and level of background noise. Further, these technologies are complex and require technical expertise to process samples and interpretation of test results.

Thus, there is a need to develop technologies for detection and identification of pathogens and antibiotic resistance genes that is comprehensive, user-friendly, reduces contamination from non-pathogenic materials, is cost-effective, provides therapy-relevant results in a clinically critical timeframe and minimizes technical expertise for analysis.

### Objects

The principal object of the embodiments disclosed herein is to provide methods and systems for detection and identification of pathogens and antibiotic resistance genes (ARGs).

An object of the embodiments disclosed herein is to provide a pre-sequencing method for enriching target nucleic acid regions for improving sensitivity of the method.

An object of the embodiments disclosed herein is to provide a high throughput, rapid and comprehensive method for identification of pathogens in a subject.

An object of the embodiment herein is to provide a method that is capable of rapidly analyzing sequence data with results in less than 10 hours.

An object of the embodiments disclosed herein is to provide a method for identification of co-infections of different pathogens in a sample.

An object of the embodiments disclosed herein is to provide a single test for identification of pathogen at species level, capable of detecting multiple pathogen classes (bacteria, fungi, difficult/unculturable anaerobes, viruses, parasites etc.) and several antibiotic resistant genes (ARGs).

Another object of the embodiments disclosed herein is to provide a method that enables doctors to take early informed actions based on the results thereby improving patient care and disease outcomes.

Another object of the embodiments herein is to provide a method that includes using sequence specific primers, performing genomic analysis and interpreting the analysis in context of relevant metadata.

Another object of the embodiments disclosed herein is to provide a method that is user friendly and automated analysis that does not require specialized bioinformatics and computational skills.

Another object of the embodiments disclosed herein is to provide a kit for pre-sequencing processing of sample for identification and detection of pathogens and antibiotic resistant genes.

These and other objects of the embodiments herein will be better appreciated and understood when considered in conjunction with the following description and the accompanying drawings. It should be understood, however, that the following descriptions, while indicating preferred embodiments and numerous specific details thereof, are given by way of illustration and not of limitation. Many changes and modifications may be made within the scope of the embodiments herein without departing from the spirit thereof, and the embodiments herein include all such modifications.

### BRIEF DESCRIPTION OF DRAWINGS

The embodiments disclosed herein are illustrated in the accompanying drawings. The embodiments herein will be better understood from the following description with reference to the drawings, in which:

FIG. 1 provides a flowchart depicting pre-sequencing according to embodiments as disclosed herein;

FIG. 2 depicts a reporting platform for generating a report based on a genomic analysis of a received sample, according to embodiments as disclosed herein; and

FIG. 3 is a flowchart depicting a process for generating a report, according to embodiments as disclosed herein.

### DETAILED DESCRIPTION

The embodiments herein and the various features and advantageous details thereof are explained more fully with reference to the non-limiting embodiments that are illustrated in the accompanying drawings and detailed in the following description. Descriptions of well-known components and processing techniques are omitted so as not to unnecessarily obscure the embodiments herein. The examples used herein are intended merely to facilitate an understanding of ways in which the embodiments herein may be practiced and to further enable those of skill in the art to practice the embodiments herein. Accordingly, the examples should not be construed as limiting the scope of the embodiments herein.

For the purposes of interpreting this specification, the following definitions will apply and whenever appropriate the terms used in singular will also include the plural and vice versa. It is to be understood that the terminology used herein is for the purposes of describing particular embodiments only and is not intended to be limiting. The terms “comprising”, “having” and “including” are to be construed as open-ended terms unless otherwise noted.

The embodiments herein disclose a method for performing genomic analysis. The method, according to embodiments herein, is used for detection and identification of pathogens in a subject or any sample obtained therefrom. The method according to embodiments herein includes pre-sequencing, by a pre-processing module, to obtain a polynucleotide library for sequencing, wherein the pre-sequencing is conducted by combining sequence specific primers; performing sequencing, by the pre-processing module, by loading the polynucleotide library on a sequencing platform to obtain raw sequence data; performing, by a genomic analysis module, basecalling and demultiplexing on the raw sequencing data to generate a first level of sequence data; generating, by the genomic analysis module, the first quality filtering for the first level of the sequence data; filtering, by the genomic analysis module, irrelevant reads of the sequence data from the first level of sequence data by removing irrelevant reads that are below a pre-defined quality threshold; classifying, by the genomic analysis module, potential reads of interest based on presence of primers and length of reads in the filtered data, which can be based on the presence of the sequence specific primers; performing, by the genomic analysis module, annotation of the classified data; interpreting, by the genomic analysis module, the annotated data based on sequencing level, quality thresholds, threshold of findings, and metadata; and generating, by a report generation module, a report, based on the interpreted data and epidemiological context, wherein the report can designate one or more pathogen genus/species and Antibiotic Resistant Genes (ARG) identification.

Embodiments herein include pre-sequencing, said pre-sequencing includes obtaining a sample, extracting nucleic acid molecules from the sample; enriching a plurality of target nucleic acid regions present in the sample by combining sequence specific primers to obtain targeted amplicons; and processing the targeted amplicons to obtain a polynucleotide library for sequencing.

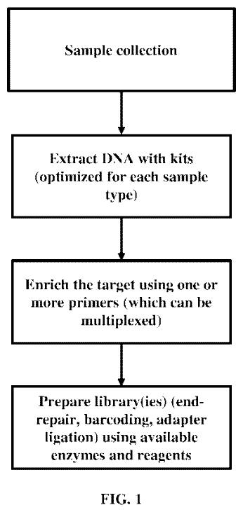

FIG. 1 provides a flowchart depicting pre-sequencing, according to embodiments as disclosed herein.

In an embodiment pre-sequencing includes obtaining a sample. The term “sample” as used herein, refers to any material, having or suspected of having pathogens or pathogenic material causing or capable of causing infections such as nucleic acid molecules, e.g.: DNA and/or RNA, proteins, peptides, etc. It includes a diagnostic specimen that is withdrawn, derived or otherwise obtained from a subject and processed for diagnostic testing. Non limiting examples of sample includes aseptic body fluids such as whole blood, plasma, serum, Cerebrospinal fluid (CSF) or any fluid aspirate or tissue extracted from human subject, pus, Bronchoalveolar Lavage (BAL) sample, pleural fluid; non sterile body samples such as respiratory samples, sputum, urine, stool, mucus, saliva; tissue abscess, wound drainage, and so on. In another embodiment, the sample includes any sample that is generally used for detection of infection e.g.: sepsis, fever of unknown origin, suspected bloodstream infection, urinary tract infection, respiratory infection. In yet another embodiment, the sample may include culture-based microbial sample, such as, but not limited to, bacterial samples such as aerobic bacteria, anaerobic bacteria, gram positive bacteria, gram negative bacteria, and bacteria that are difficult-to-culture; other pathogens such as fungi, parasites, protozoa; and combinations thereof. In an embodiment, the sample is whole blood. In another embodiment, the sample is plasma. In yet other embodiment, the sample is one comprising extracted nucleic acid molecules or nucleotides e.g.: DNA. In another embodiment, the sample is one comprising genomic and/or pathogenic DNA, or genetic material of pathogen(s). In another embodiment, the sample is a culture-based microbial sample.

The sample, according to the embodiments herein, may be collected, prepared or enriched or optionally processed. The term “optionally processed”, as used herein, refers to the steps performed to modify a sample before diagnostic testing. In an embodiment, processing comprises collecting, separating, removing a portion of the sample, and/or adding an additional component, such as a protease, to the sample. The optimization of sample preparation is crucial for high-quality sequencing results and specific considerations are necessary for different matrices. For example, detecting low-titer pathogens in clinically relevant samples is complicated by a high level of host genetic material. Accordingly, embodiments herein may also include enriching sample by removing host cells. The term, “host cells”, as used herein, refers to, cells derived from the host or subject that are distinct from commensal (e.g., microbial cells which are part of the host microbiome), infectious (e.g., pathogenic microbes) or contaminating cells (e.g., introduced accidentally during sample collection or preparation). The term “host” may refer to a patient or medical subject. Various generally known methods of sample collection, enriching, or processing, would be apparent to a person skilled in the art.

The term “subject”, as used herein, includes any subject having or suspected of having an infection. In an embodiment, infection refers to any infection that may lead to or cause or is suspected of causing sepsis or sepsis like condition, e.g.: bacterial infection, fungal infection, etc. The term “sepsis”, as used herein, includes related terms such as systemic inflammatory response syndrome (SIRS), septicemia, septic shock, etc. Subject refers to mammals such as bats, pigs, mice, rats, dogs, cattle and humans, particularly humans. In an embodiment, subject is an individual having or suspected of infection caused by pathogen. In another embodiment, subject is an individual having or suspected of having sepsis causing pathogen or pathogen material. In another embodiment, subject is an individual showing symptom of sepsis. In yet other embodiment, subject is an individual having or suspected of having sepsis. In an embodiment, infection refers to any infection that may lead to or cause infection e.g.: bacterial infection, fungal infection, fever of unknown origin, etc. In another embodiment, subject is an individual having or suspected of having infection causing pathogen or pathogen material. In another embodiment, subject is an individual showing symptom of infection.

The term, “obtaining”, as used herein, refers to either withdrawing a clinical sample from a subject or receiving a clinical sample which has been withdrawn from a subject. Withdrawing a clinical sample from a subject may be achieved by any route known in the art, including, but not limited to: intravenous, intra-arterial, intraperitoneal, intracranial, intra-spinal, intramuscular, intra-urethral, intra-tracheal, and intra-nasal. Withdrawing may be achieved by using a syringe, biopsy needle, aspiration tube, swab or similar devices, or by urination, expectoration, and wound drainage.

In an embodiment, pre-sequencing includes extracting nucleic acid molecules from the sample. In an example herein, the nucleic acid is DNA. Nucleic acid extraction may be achieved by techniques generally known in the art, including but not limited to, Proteinase K, Phenol-chloroform isoamyl alcohol, CTAB method, spin column-based methods and magnetic bead-based technique. Embodiments herein, may use any of such generally known methods for extracting DNA. DNA extraction may also be carried out using commercially available reagents/kits such as, but not limited to, Qiagen® (QIAamp®, DNEasy®), Roche Applied Science (MagNA Pure kits), Epicentre® (Masterpure™ kits), etc. In an embodiment, DNA extraction method may be optimized according to sample type. All such methods and modifications of such methods are understood to be included within the scope of the embodiments herein. The embodiments herein include sequencing of nucleotide sequences on NGS platforms, therefore, it is understood that the extracted nucleic acid molecules may be processed by methods generally known in the art, to achieve a suitable nucleic acid sample for sequencing using NGS platforms. In an embodiment, the nucleic acid sample is suitable for nucleotide sequencing on NGS platforms.

Pre-sequencing, according to embodiments herein further includes enriching a plurality of target nucleic acid regions present in the sample by combining sequence specific primers to obtain targeted amplicons.

The term “enriching”, as used herein refers to a process for amplifying or multiplying a plurality of target nucleic acid regions in a sample. In an embodiment, the target nucleic acid regions are specific sequences in microbial genome. Further in an embodiment, the target microbial nucleic acid region is from a pathogen, such as bacterium, fungal or viral.

In an embodiment, the target microbial nucleic acid regions are enriched using an amplification technique. Non-limiting examples of amplification techniques include amplicon-based method such as polymerase chain reaction (PCR), multiplex PCR, Real-time PCR, Nested PCR, Droplet-based digital PCR, colony PCR, using molecular baits and so on. The irrelevant nucleic acid may selectively be removed or degraded. In an embodiment, the target microbial nucleic acid regions are enriched using polymerase chain reaction-based amplification technique. In another embodiment, the target microbial nucleic acid regions are enriched using multiplex PCR based amplification technique. Multiplex PCR is a technique for simultaneous amplification and detection of multiple targets in a single reaction well, with a different pair of primers for each target. Enriching target nucleic acid region ensures that sequencing is focused to predominantly screen target regions of interest with minimal off-target sequencing, making it more accurate, sensitive and economical.

Amplification of a population of nucleic acids by any of the previously-mentioned methods requires a primer and a polymerase. The term “primer”, as used herein, refers to its generally known meaning in the art. Primers refer to nucleotide strands capable of hybridizing with a polynucleotide sequence or a target nucleotide sequence and is capable of providing a point of initiation for complementary nucleotide strand synthesis. The term “sequence specific primer”, as used herein, refers to any polynucleotide sequence of interest present in a target nucleic acid region, for e.g.: polynucleotide sequences of one or more pathogens, antibiotic resistance gene, etc. In an embodiment, the target nucleotide region is a gene sequence of a pathogen e.g.: bacteria, fungi, protozoa, or pathogen causing or capable of causing infection in a subject. In an embodiment, the target nucleotide region is an antibiotic resistance gene of a pathogen.

In an embodiment, pre-sequencing includes amplifying target nucleic acid regions present in a sample by introducing at least one primer sequence selected from a group consisting of SEQ ID NO. 1 to SEQ ID NO. 10. In an embodiment, the reported primer sequences are sequence specific primer sequences. The reported primer sequences include primers having SEQ ID NO. 1 to SEQ ID NO. 10 (Table 1).

Table 1 provides list of reported primer sequences
(SEQ ID NO. 1 to SEQ ID NO. 10) used for amplification
of target nucleic acids regions.

| | Target Gene/ | | |
|---|---|---|---|
| Sr No. | region | PMID | Sequence Number (IDs) |
| 1. | blaTEM_F | 26104141 | Sequence Number (ID): 1 |
| 2. | blaTEM_R | 26104141 | Sequence Number (ID): 2 |
| 3. | blaSHV_F | 26104141 | Sequence Number (ID): 3 |
| 4. | blaSHV_R | 26104141 | Sequence Number (ID): 4 |
| 5. | OXA-1_F | 26104141 | Sequence Number (ID): 5 |
| 6. | OXA-1_R | 26104141 | Sequence Number (ID): 6 |
| 7. | ITS_F | 24933453 | Sequence Number (ID): 7 |
| | (Fungal ITS) | | |
| 8. | ITS_R | 24933453 | Sequence Number (ID): 8 |
| | (Fungal ITS) | | |
| 9. | 16S_F | 30961521 | Sequence Number (ID): 9 |
| | (Bacterial 16s) | | |
| 10. | 16S_R | 30961521 | Sequence Number (ID): 10 |
| | (Bacterial 16s) | | |

In an embodiment, pre-sequencing includes subjecting the extracted nucleic acid e.g., pathogen nucleic acid to amplification technique. In an exemplary embodiment, nucleic acid may be normalized and then subjected to PCR tubes containing primer mix along with other PCR components such as DNA polymerase, deoxynucleotides (dNTPs), etc., to obtain targeted amplicons.

In an embodiment, PCR amplification is carried out with an initial cycle of heat activation at 94 to 98 degrees Celsius for 2 to 6 minutes; 25 cycles of denaturation at 94 to 98 degrees Celsius for 20 to 35 seconds; 25 cycles of annealing at 60 to 65 degrees Celsius for 20 to 35 seconds; 25 cycles of extension at 65 to 72 degrees Celsius for 2 to 3 minutes; final extension at 65 to 72 degrees Celsius for 5 to 7 minutes; and finally holding the reaction at 4 degrees Celsius.

In an embodiment, PCR amplification is carried out with an initial cycle of heat activation at 98 degrees Celsius for 3 minutes; 25 cycles of denaturation at 98 degrees Celsius for 30 seconds; 25 cycles of annealing at 62 degrees Celsius for 30 seconds; 35 cycles of extension at 72 degrees Celsius for 2 minutes; final extension at 72 degrees Celsius for 5 minutes; and finally holding the reaction at 4 degrees Celsius.

In an embodiment, the sequence specific primers are capable of achieving amplification of pathogenic bacterial nucleic acid. In yet other embodiment, the sequence specific primers are capable of achieving amplification of pathogenic fungal nucleic acid. In yet other embodiment, the sequence specific primers are capable of achieving amplification of one or more antibiotic resistant genes.

Pre-sequencing further includes processing the targeted amplicons to obtain a polynucleotide library for sequencing. The term “polynucleotide library” or “sequencing library”, as used herein includes DNA fragments of a defined length distribution with oligomer adapters at the 5′ and 3′ end for barcoding.

In an embodiment, the targeted amplicons are further processed for preparing sequencing library. Preparing sequencing library, according to embodiments herein, includes end repair of targeted amplicons, adapter ligation and barcoding. The term “adapter” and “barcode”, as used herein refers, to its generally known meaning in the art. Adapters are commonly used in nucleotide library creation and facilitate high throughput sequencing of polynucleotide or target sequences, particularly by attaching or ligating the adapter sequence to the polynucleotide or target sequence. Adapters are non-target polynucleotide sequences which may be tagged or untagged and may be appended to one or both ends of polynucleotide or target sequences. Further, adapters may, optionally, include one or more primer binding sites. In some embodiments, it refers to a mix of adapter sequences intended for ligation to polynucleotide sequences of pathogens, e.g.: infection causing pathogens, at one or both ends. The term “Barcodes”, as used herein, includes related terms such as “barcode sequences”, “barcode tags”, “molecular barcodes”, etc. It refers to short polynucleotide sequences that are used to identify specific sample, polynucleotide or target sequences or group thereof in a pool of samples. In general, barcodes are used to label samples which facilitate pooling of multiple samples to achieve sequencing in high throughput. Barcoding refers to attaching or ligating a barcode sequence to each DNA fragment, within a given sample. Multiple unique barcodes may be used to identify or differentiate multiple samples. It includes tagged and untagged molecules which are capable of facilitating sample identification. The size of barcode and adapter sequences may vary from 10 nucleotides to 100 nucleotides or more. Barcodes are generally shorter sequences and may also in some cases be included within the adapter sequence. Generally, adapters and barcodes are predetermined or known sequences of synthetic or natural origin and may be single or double stranded depending on the requirement/sequencing platforms used. Various methods are generally known, and may be used in embodiments herein, to attach adapters/barcodes to polynucleotide/target sequences. Such sequences are commercially available which may be used in various embodiments herein.

In an embodiment, the targeted amplicons are treated with end repair enzyme mix to obtain blunt ended polynucleotide strands. In general, End Repair Enzyme Mix comprises of an optimized mixture of T4 DNA Polymerase and Klenow Fragment for achieving effective blunting of fragmented DNA, and T4 Polynucleotide Kinase for efficient phosphorylation of DNA ends. The end repair enzyme mix, and targeted amplicon mixture is incubated in a thermal cycler for a suitable time period which may include incubating the mixture at 20 degrees Celsius for 5 to 30 minutes followed by heating at 65 degrees Celsius for 5-30 minutes.

The blunt ended polynucleotide strands obtained after the end repair step are further subjected to a barcode ligation step. The barcode ligation step includes mixing suitable volume of the blunt ended polynucleotide strands with a unique barcode and incubating said mixture in a thermal cycler for 20 degrees Celsius for 5 to 30 minutes followed by heating at 65 degrees Celsius for a period of 5 to 30 minutes. The barcode amplicons thus obtained are subsequently purified using magnet assisted purification beads. In an example herein, the purification technique includes pooling barcoded tubes; adding purification beads to the pooled barcoded tubes followed by vortexing for 10 minutes at room temperature; spinning and pelleting the beads on a magnetic stand for 5 minutes and discarding the supernatant. The beads are then washed using a purification buffer, kept on a magnetic rack and allowed to pellet until a clear and colorless elute is obtained. The previous step is repeated, and the beads are again washed, and the tube is kept on a magnet followed by addition of 80% ethanol. Ethanol is removed, and the tube is kept for drying for a few seconds. The tube is removed from the magnetic stand; resuspending pellet in nuclease free water followed by 2 minutes followed by incubation at room temperature. The beads are then pelleted on a magnet until a clear and colorless elute is obtained and finally the eluted sample is quantified using a suitable nucleic acid quantifying instrument, such as the Qubit fluorometer.

This step is followed by attaching adapters to at least one end of barcoded amplicons and purifying adapter bound barcoded amplicons using magnet assisted purification beads. After purification, the adapter bound barcoded amplicons or polynucleotide library may further be diluted and processed for loading on sequencing platform. The sequencing library preparation steps may further include various modifications so as to make the process faster.

In an embodiment, the genomic analysis method further comprises performing sequencing by loading the polynucleotide library on suitable sequencing platform to obtain raw sequence data. Suitable next generation sequencing (NGS) platform may be used to obtain sequence data. The term “sequencing” as used herein, refers to a method of sequence determination. It includes technologies used to determine the sequence of a biomolecule, e.g., a nucleic acid such as DNA or RNA. In an embodiment, sequencing includes next generation sequencing methods. Examples of sequencing methods include, but are not limited to, sequencing-by-synthesis (SBS) (Illumina sequencing), single-molecule Real Time sequencing (SMRT) (PacBio), oxford nanopore sequencing (e.g., MinION), sequencing-by-ligation, sequencing-by-hybridization, solexa sequencing (Illumina), Digital Gene Expression (Helicos), Next generation sequencing (e.g., Roche 454, Solexa platforms such as HiSeq2000, and SOLID), Single Molecule Sequencing by Synthesis (SMSS) (Helicos), massively-parallel sequencing, shotgun sequencing and Maxim-Gilbert sequencing. In one embodiment, sequencing includes nanopore sequencing method. Nanopore sequencing or oxford nanopore sequencing (ONT) is a next generation sequencing technology that enables direct, real-time analysis of long DNA or RNA fragments. Typically, the technology uses a protein nanopore, wherein changes to an electrical current as nucleic acids pass through the nanopore are detected. The resulting signal is decoded to provide the specific DNA or RNA sequence. Although ONT has a very high (−10%) per base error rate, it is highly portable and deployable in field conditions, and furthermore sequencing reads are reported real-time. In an embodiment, the method is compatible with any suitable next generation sequencing technique. In another embodiment, the sequencing is performed using nanopore assisted sequencing technique. In yet another embodiment, sequencing is performed by using a sequencing technology wherein long reads are generated.

Post-sequencing, a plurality of sequence data, also known as sequence reads, are obtained from the sequencing library. In NGS, data or read refers to the number of base pairs sequenced from a DNA fragment. In an embodiment, the reads are obtained as raw FASTQ/FAST5 file. The sequencing reads are further used as input for performing genomic analysis.

Basecalling and demultiplexing is performed on the sequencing reads to generate a first level of sequence data. In an embodiment herein, the basecalling can be performed using relevant available tools. Non-limiting examples of tools that can be used for performing basecalling can be, but not limited to, Albacore, Guppy, bcl2fastq, and so on. In an embodiment herein, the demultiplexing can be performed using relevant available tools. The first level of the sequence data can be quality scored to generate a first quality score. In an embodiment herein, the quality scoring can be performed using relevant available tools. Non-limiting examples of tools that can be used for performing quality scoring can be, but not limited to, Guppy, Nanofilt, Trimmomatic, fastp and so on. Based on the generated first quality score, irrelevant reads of the sequence data can be filtered from the first level of sequence data. This can involve removing irrelevant reads with quality scores that are below a pre-defined threshold. In an example herein, the pre-defined threshold can be defined in terms of Phred or Q score. Examples of the irrelevant reads that can be removed here can be, but not limited to, basis length, reads with low quality, chimeric sequences, host DNA (filtering of human reads), high-N-content reads and repeated sequences, DNA over-amplification, eliminating reads corresponding to barcodes, and adapters ligated to the DNA fragment for the sequencing purpose and primers used for the amplification. Further, from the filtered data, irrelevant reads and sequences are filtered out. Based on the presence of primer(s) (which were used for target enrichment) and length of reads, potential reads of interest are classified. The filtered data can be quality scored to generate a second quality score. In an embodiment herein, the quality scoring can be performed using relevant available tools. The filtered data is further processed for annotation. Annotation may be performed using alignment tools, consensus sequences, sequence similarity, GC content, average nucleotide identity, maximum parsimony, maximum likelihood, relative distance, cladistic single nucleotide polymorphisms, and time to most recent common ancestor. Multiple alignment or assembly strategies may be used as are known in the art. The generated alignments or consensus sequences are further annotated using various reference databases that are known in the art. Non-limiting examples of microbial genome databases include National Center for Biotechnology Information (NCBI) Reference Sequence (RefSeq) genome database, Pathosystems Resource Integration Center (PATRIC), Comprehensive Antibiotic Resistance Database (CARD), National Database of Antibiotic Resistant Organisms (NDARO) and so on. In an embodiment herein, the annotation can comprise performing pathogen annotation using a custom database, wherein this comprises size matching, matching of sequences to target pathogen regions delineating information such as pathogen genus, species or antibiotic resistance genes, and so on. In an embodiment herein, the annotation can comprise performing ARG annotation using a custom database. In an embodiment, the method is capable of identifying pathogens such as bacteria, fungi, parasites and viruses. Further in an embodiment, the method is also capable of semi-quantitative detection of pathogens present in a sample.

The annotated data can be interpreted based on factors such as, but not limited to, sequencing level, quality thresholds, threshold of findings, metadata (patient metadata (age, gender, location, movement history, and so on), clinical criteria (clinical history, current complaints, physical signs of infection (such as, fever, inflammation, respiratory distress, altered mental status, and so on), number of days that the user has been suffering from the symptoms, number of days that the user has been hospitalized, symptoms related to detected pathogens, other prescribed tests, and so on), treatment related criteria (medicines already being taken by the patient, provisional diagnosis, past and current therapy, antibiotics already administered to the user, and so on), laboratory criteria (complete blood count (CBC), Erythrocyte Sedimentation Rate (ESR), biochemical markers, culture tests, and so on), biomarker information (infectious vs non-infectious pathogen >bacterial vs fungal vs virus or polymicrobial infections)), and so on.

A report can be generated based on the interpreted data and additional parameters (such as, but not limited to, epidemiological context (epidemiology review, local or global outbreaks, possible infection, known co-infections or co-morbidities, general public health data, and so on). The report can be based on clinical relevance (wherein the reports use the metadata for clinical decision(s)). The report can designate one or more pathogen genus/species and/or ARG identification. The report can include a score, wherein the score can be used to identify presence or absence of disease. The score may be generated based on one or more pre-decided criteria, decision matrix, Machine Learning (ML) models, etc., and a respective weightage assigned to each of the pre-decided criteria. The weightages can vary with the user, and can depend on the metadata. The score can indicate the probability of infection in the patient. The report can be provided to an authorized user (such as a clinician, patient, doctor, and so on) using a user interface. In an embodiment, data is analyzed and genome analysis report is generated within a period of 5 to 25 minutes. The system may also be integrated into a web application that can be viewed by a user.

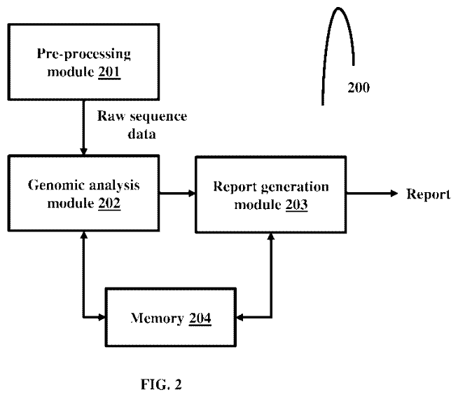

FIG. 2 depicts a reporting platform for generating a report based on a genomic analysis of a received sample, according to embodiments as disclosed herein. The reporting platform 200, as depicted, comprises a pre-processing module 201, genomic analysis module 202, a report generation module 203, and a memory 204. In an embodiment herein, the genomic analysis module 202 may be cloud-based or edge-based. In an embodiment herein, the report generation module 203 may be accessed through the cloud or the edge.

The memory 204 stores at least one of, the custom database, extracted DNA, input raw sequence data, metadata, the first and second quality, annotated data, interpretations, epidemiological contexts, generated reports, and so on. Examples of the memory 204 can be, but are not limited to, NAND, embedded Multimedia Card (eMMC), Secure Digital (SD) cards, Universal Serial Bus (USB), Serial Advanced Technology Attachment (SATA), Solid-State Drive (SSD), the cloud, a data server, a file server, and so on. Further, the memory 204 can include one or more computer-readable storage media. The memory 204 can include one or more non-volatile storage elements. Examples of such non-volatile storage elements can include Read Only Memory (ROM), magnetic hard discs, optical discs, floppy discs, flash memories, or forms of Electrically PROgrammable Memories (EPROM) or Electrically Erasable and PROgrammable Memories (EEPROM). In addition, the memory 204 can, in some examples, be considered a non-transitory storage medium. The term “non-transitory” can indicate that the storage medium is not embodied in a carrier wave or a propagated signal. However, the term “non-transitory” should not be interpreted to mean that the memory is non-movable. In certain examples, a non-transitory storage medium can store data that can, over time, change (e.g., in Random Access Memory (RAM) or cache).

The pre-processing module 201 can communicate with one or more external modules/devices (such as thermocycler/PCR Machine/DNA Amplifier; sequencing machines, etc.), which enable the pre-processing module 201 to perform its functions. The pre-processing module 201 can include one or more functions/tasks such as obtaining a sample; extracting nucleic acid molecules from the sample; enriching a plurality of target nucleic acid regions present in the sample by combining sequence specific primers to obtain targeted amplicons; and processing the targeted amplicons to obtain a polynucleotide library for sequencing; and performing sequencing to obtain raw sequence data.

The term ‘genomic analysis module 202,’ as used in the present disclosure, can refer to, for example, hardware including logic circuits; a hardware/software combination such as a processor executing software; or a combination thereof. For example, the processing circuitry more specifically can include, but is not limited to, a central processing unit (CPU), an arithmetic logic unit (ALU), a digital signal processor, a microcomputer, a field programmable gate array (FPGA), a System-on-Chip (SoC), a programmable logic unit, a microprocessor, application-specific integrated circuit (ASIC), etc. For example, the genomic analysis module 202 can include at least one of, a single processer, a plurality of processors, multiple homogeneous or heterogeneous cores, multiple Central Processing Units (CPUs) of different kinds, microcontrollers, special media, and other accelerators. In an embodiment herein, the genomic analysis module 202 can be a dedicated module. In an embodiment herein, the genomic analysis module 202 can be a generic module, which can perform one or more other functions/tasks, in addition to embodiments as disclosed herein.

Raw sequence data obtained from pre-processing module 201 can be the input to the genomic analysis module 202. The genomic analysis module 202 can perform basecalling and demultiplexing on the input sequencing reads to generate a first level of sequence data. In an embodiment herein, the genomic analysis module 202 can perform basecalling using relevant available tools. In an embodiment herein, the genomic analysis module 202 can perform demultiplexing using relevant available tools. The genomic analysis module 202 can generate a first quality score for the first level of the sequence data. In an embodiment herein, the genomic analysis module 202 can generate the first quality score using relevant available tools. Based on the generated first quality score, the genomic analysis module 202 can filter the irrelevant reads of the sequence data from the first level of sequence data. This can involve the genomic analysis module 202 removing irrelevant reads with quality scores that are below a pre-defined quality score threshold. Examples of the irrelevant reads that can be removed here can be, but not limited to, basis length, reads with low quality, chimeric sequences, host DNA (filtering of human reads), high-N-content reads and repeated sequences, DNA over-amplification, eliminating reads corresponding to barcodes, and adapters ligated to the DNA fragment for the sequencing purpose and primers used for the amplification.

Further, from the filtered data, the genomic analysis module 202 can filter out the irrelevant reads and sequences. Further from the filtered data, the genomic analysis module 202 can classify potential reads of interest based on the presence of primer(s) (which were used for target enrichment) and length of reads. The genomic analysis module 202 can generate the second quality score for the filtered data. In an embodiment herein, the genomic analysis module 202 can generate the second quality score using relevant available tools. The genomic analysis module 202 can perform annotation on the filtered data. In an embodiment herein, the genomic analysis module 202 can perform annotation using methods such as, but not limited to, sequence alignment, sequence assembly, contiguous sequence similarity, consensus, sequence similarity, GC content, average nucleotide identity, maximum parsimony, maximum likelihood, relative distance, cladistic single nucleotide polymorphisms, and time to most recent common ancestor. The generated alignments or consensus sequences can be further annotated using various reference databases that are known in the art. In an embodiment herein, the annotation can comprise the genomic analysis module 202 performing pathogen annotation using a custom database, wherein this comprises size matching, matching of sequences to target pathogen regions delineating information such as pathogen genus, species or antibiotic resistance genes, and so on. In an embodiment herein, the annotation can comprise the genomic analysis module 202 performing ARG annotation using a custom database.

The genomic analysis module 202 can interpret the annotated data based on factors such as, but not limited to, sequencing level, quality thresholds threshold of findings, metadata (patient metadata (age, gender, location, movement history, and so on), clinical criteria (clinical history, current complaints, physical signs of infection (such as, fever, inflammation, respiratory distress, altered mental status, and so on), number of days that the user has been suffering from the symptoms, number of days that the user has been hospitalized, symptoms related to detected pathogens, other prescribed tests, and so on), treatment related criteria (medicines already being taken by the patient, provisional diagnosis, past and current therapy, antibiotics already administered to the user, and so on), laboratory criteria (complete blood count (CBC), Erythrocyte Sedimentation Rate (ESR), biochemical markers, culture tests, and so on), biomarker information (infectious vs non-infectious pathogen >bacterial vs fungal vs virus or polymicrobial infections)), and so on. The genomic analysis module 202 can send the interpreted data including the metadata to the report generation module 203.

The term ‘report generation module 203,’ as used in the present disclosure, can refer to, for example, hardware including logic circuits; a hardware/software combination such as a processor executing software; or a combination thereof. For example, the processing circuitry more specifically can include, but is not limited to, a central processing unit (CPU), an arithmetic logic unit (ALU), a digital signal processor, a microcomputer, a field programmable gate array (FPGA), a System-on-Chip (SoC), a programmable logic unit, a microprocessor, application-specific integrated circuit (ASIC), etc. For example, the report generation module 203 can include at least one of, a single processer, a plurality of processors, multiple homogeneous or heterogeneous cores, multiple Central Processing Units (CPUs) of different kinds, microcontrollers, special media, and other accelerators. In an embodiment herein, the report generation module 203 can be a dedicated module. In an embodiment herein, the report generation module 203 can be a generic module, which can perform one or more other functions/tasks, in addition to embodiments as disclosed herein.

The report generation module 203 can generate a report based on the interpreted data and additional parameters (such as, but not limited to, epidemiological context (epidemiology review, local or global outbreaks, possible infection, known co-infections or co-morbidities, general public health data, and so on). The report generation module 203 can base the report on clinical relevance (wherein the reports use the metadata for clinical decision(s)). The report can designate one or more pathogen genus/species and/or ARG identification. The report, as generated by the report generation module 203, can include a score, wherein the score can be used to identify presence or absence of disease. The report generation module 203 may generate the score based on one or more pre-decided criteria, decision matrix, Machine Learning (ML) models, etc. The score can indicate the probability of infection in the patient. The report generation module 203 may also generate the report in the form of graphical representation. The report generation module 203 can provide the report to an authorized user (such as a clinician, patient, doctor, and so on) using a user interface. The report generation module 203 can store the report in a location, such as, but not limited to the memory 204, the Cloud, a remote server, a local server, and so on.

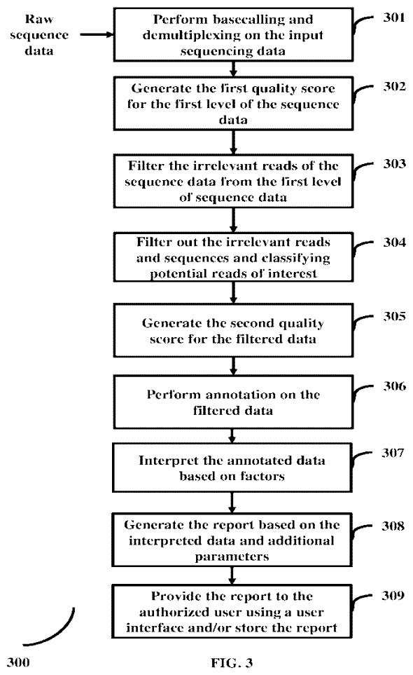

FIG. 3 is a flowchart depicting a process for generating a report, according to embodiments as disclosed herein. Raw sequence data is provided as input to the reporting platform 200. In step 301, the reporting platform 200 performs basecalling and demultiplexing on the input sequencing reads to generate a first level of sequence data, using relevant available tools. In step 302, the reporting platform 200 generates the first quality score for the first level of the sequence data using relevant available tools. Based on the generated first quality score, in step 303, the reporting platform 200 filters the irrelevant reads of the sequence data from the first level of sequence data by removing irrelevant reads with quality scores that are below a pre-defined quality score threshold. Examples of the irrelevant reads that can be removed here can be, but not limited to, basis length, chimeras, barcodes, adapter, and so on. From the filtered data, in step 304, the reporting platform 200 can filter out the irrelevant reads and sequences and classifying potential reads of interest basis presence of primers and length of reads, which can be based the presence of the primer (which was used for target enrichment). In step 305, the reporting platform 200 generates the second quality score for the filtered data using relevant available tools. In step 306, the reporting platform 200 performs annotation on the filtered data using methods such as, but not limited to, sequence alignment, sequence assembly, contiguous sequence, consensus, sequence similarity, GC content, average nucleotide identity, maximum parsimony and maximum likelihood. In an embodiment herein, the annotation comprises performing pathogen annotation using a custom database, wherein this comprises size matching, matching of sequences to target pathogen regions delineating information such as pathogen genus, species or antibiotic resistance genes, and so on. In an embodiment herein, the annotation comprises performing ARG annotation using a custom database. In step 307, the reporting platform 200 interprets the annotated data based on factors such as, but not limited to, sequencing level, quality thresholds threshold of findings, metadata (patient metadata (age, gender, location, movement history, and so on), clinical criteria (clinical history, current complaints, physical signs of infection (such as, fever, inflammation, respiratory distress, altered mental status, and so on), number of days that the user has been suffering from the symptoms, number of days that the user has been hospitalized, symptoms related to detected pathogens, other prescribed tests, and so on), treatment related criteria (medicines already being taken by the patient, provisional diagnosis, past and current therapy, antibiotics already administered to the user, and so on), laboratory criteria (complete blood count (CBC), Erythrocyte Sedimentation Rate (ESR), biochemical markers, culture tests, and so on), biomarker information (infectious vs non-infectious pathogen >bacterial vs fungal vs virus or polymicrobial infections)), and so on. In step 308, the reporting platform 200 generates the report based on the interpreted data and additional parameters (such as, but not limited to, epidemiological context (epidemiology review, local or global outbreaks, possible infection, known co-infections or co-morbidities, general public health data, and so on). The reporting platform 200 bases the report on clinical relevance (wherein the reports use the metadata for clinical decision(s)), wherein the report can designate one or more pathogen genus/species and/or ARG identification. The report can include the score used for identifying presence or absence of disease, wherein the score can indicate the probability of infection in the patient. The reporting platform 200 generates the score based on one or more pre-decided criteria, decision matrix, Machine Learning (ML) models, etc. The report generation module 203 may also generate the report in the form of graphical representation. In step 309, the reporting platform 200 provides the report to the authorized user (such as a clinician, patient, doctor, and so on) using a user interface and/or store the report in a location, such as, but not limited to the memory 204, the Cloud, a remote server, a local server, and so on. The various actions in method 300 may be performed in the order presented, in a different order or simultaneously. Further, in some embodiments, some actions listed in FIG. 3 may be omitted.

It is understood that various minor modifications to the method may be apparent to a person skilled in the art without departing from the spirit and scope of the invention disclosed in various embodiments herein. All such modifications are understood to be included within the scope of this invention.

The embodiments disclosed herein provide a method for genomic analysis. The method, according to various embodiments herein, is useful in detection and identification of pathogens such as bacteria, fungi and/or protozoa in samples. In an embodiment, the method is also useful for detection and identification of sepsis causing pathogens. While pathogenic sepsis is generally known to be caused by bacterial infections, viruses and fungi may also in some cases lead to septic conditions in a subject. Embodiments herein provide a method for detection and identification of all such sepsis causing pathogens, pathogenic material, and pathogenic sepsis conditions. In an embodiment, the method is also useful in detection and identification of genes responsible for antimicrobial resistance in pathogens. In yet another embodiment, the method also facilitates identification of co-infections of different pathogens in a sample. Further, the method allows several samples to be assessed simultaneously. The method, according to embodiments herein, is capable of detecting difficult-to-culture anaerobes, and rare and emerging pathogens which could be missed by culture-based methods or are not part of targeted gene panel. The method, according to embodiments herein, provides a single test for identification of pathogen at species over 400 pathogens (bacteria, fungi, level, capable of detecting difficult/unculturable anaerobes) and over 24 antibiotic resistant genes (ARGs).

Non-limiting examples of pathogenic gram-negative bacteria that can be detected and identified using disclosed method include Acinetobacter baumannii, Acinetobacter calcoaceticus, Bacteroides fragilis, Klebsiella pneumoniae, Proteus vulgaris, Haemophilus influenzae, Cosenzaea myxofaciens, Enterobacter cloacae, Escherichia coli, Klebsiella aerogenes, Klebsiella oxytoca, Neisseria meningitidis, Proteus alimentorum, Proteus columbae, Proteus cibarius, Proteus terrae, Motiliproteus sediminis, Shimwellia pseudoproteus, Obesumbacterium proteus, Proteus hauseri, Proteus penneri, Pseudomonas aeruginosa, Salmonella enterica subsp. enterica serovar typhimurium strain, Salmonella enterica subsp. arizonae, Salmonella bongori, Salmonella enterica subsp. enterica, Salmonella enterica subsp. diarizonae, Salmonella enterica subsp. salamae, Salmonella enterica subsp. houtenae, Salmonella enterica subsp. indica, Serratia marcescens, Stenotrophomonas maltophilia, and so on.

Non-limiting examples of pathogenic gram-positive bacteria that can be detected and identified using disclosed method include Staphylococcus aureus, Enterococcus faecalis, Enterococcus faecium, Listeria monocytogenes, Staphylococcus epidermidis, Staphylococcus lugdunensis, Streptococcus agalactiae, Streptococcus pneumoniae, Streptococcus pyogenes and so on.

Non-limiting examples of pathogenic fungi that can be detected and identified using disclosed method include Candida albicans, Pichia kudriavzevii, Candida auris, Candida glabrata, Candida parapsilosis, Cryptococcus neoformans var. neoformans and so on.

Non-limiting examples of antibiotic resistant genes that can be detected and identified using disclosed method include blaTEM, blaSHV, OXA 1, OXA 48, blaVIM, blaKPC, blaNDM, CTX M 15, AmpC, OXA-181, AcOXA, GES-CPO, vanA, vanB, ermA, ermB, ermC, mupA, GES, gyrA, mcr-1, mcr-2, mecA, mecB and so on.

Embodiments herein also disclose a kit for performing disclosed method. In an embodiment, the kit comprises at least one vial of sequence specific primer mix and an instruction manual. In another embodiment, the kit comprises, at least one vial of sequence specific primer mix. The reagents, and other materials required for performing pre-sequencing and sequencing steps may depend on the type of sample and sequencing technique. The kit also includes an instruction manual for performing the particular embodiment of the kit, such as providing conditions and steps for operation of the method. The sequence specific primers provided with the kit may be suspended in an aqueous solution or as a freeze-dried or lyophilized powder.

Time for diagnosis represents a highly critical parameter in life-threatening infections such as BSI. Accordingly, the method, disclosed in various embodiments herein, is rapid with results being provided in less than 10 hours (approximately 8 hours). Further the method is cost effective and scalable such that it can be set up in any molecular diagnostics lab. The disclosed method has optimum accuracy; analytical sensitivity; analytical specificity; and concordance comparable to other generally known molecular methods. Also, the said method is user friendly as the analysis method uses generated sequence that does not require specialized bioinformatics and computational skills. The method enables doctors to take early informed actions based on the results thereby improving patient care and disease outcomes. The method, according to embodiments herein, provides end-to-end solution for pathogen diagnosis from sample to report generation.

The examples used herein are intended merely to facilitate an understanding of ways in which the embodiments herein may be practiced and to further enable those skilled in the art to practice the embodiments herein. Accordingly, the examples should not be construed as limiting the scope of the embodiments herein.

### Example 1: Sample Collection and Processing

8 ml of whole blood was collected from an adult patient (For pediatric patients 1-3 ml blood can be collected) and added into EDTA Lavender Top Tube. The tube was mixed well and centrifuged at 1500 rpm for 10 minutes at room temperature. 1 ml of supernatant was carefully transferred into multiple 1.5 ml microcentrifuge tubes until the complete supernatant was obtained. Further all the 1.5 ml microfuge tubes were centrifuged for the individual sample at 12000 rpm for 10 minutes at room temperature. 950 μl of the supernatant was discarded and around 50 μl supernatant was retained along with pellet in each tube. The contents form all the tubes was pooled with final volume of 500 μl. To this tube 180 μl of animal tissue lysis (ATL; Qiagen) buffer and 20 μl proteinase K was added and incubated at 56 degrees Celsius for 30 minutes. Further, 200 μl of buffer AL was added and mixed by vortexing for 15 s. All the mixture was transferred into the QIAamp Mini spin column and centrifuged at 8000 rpm for 1 min. The flow-through and collection tube were discarded. QIAmp Mini spin column was then placed in new 2 ml collection tube and 500 μl buffer AW1 was added to it. The tube was subsequently centrifuged at 8000 rpm for 1 min after which the flow-through and collection tube were discarded. The QIAmp Mini spin column was then placed on a fresh 2 ml collection tube and 500 μl Buffer AW2 was added and further centrifuged for 3 min at 14,000 rpm. The flow-through was discarded and the collection tube was retained. The collection tube was further centrifuged again for 1 min at 14,000 rpm. The QIAmp Mini spin column was placed in a fresh sterile 1.5 ml microcentrifuge tube and 200 μl Buffer AE was added to the tube. The tube was kept for incubation at room temperature for 1 min followed by centrifugation at 8000 rpm for 1 min for eluting the DNA. DNA concentration was determined using Qubit fluorometer.

### Example 2: Metadata Entry and Pre-Sequencing Processing

Before pre-sequencing, metadata for each sample was added. The metadata included information such as Barcode ID, Sample ID, Patient ID, sample source, collection date, library date, run date, Flowcell ID, batch number, study type, client ID, Primer, Sample count, Patients details such as name, age, gender, address, contact, doctor, etc.

For pre-sequencing process, firstly extracted DNA was normalized to 1 to 3 ng/ul. 5 μl of normalized DNA sample was then added to the respective 8 well PCR tubes containing 5 μl Master Mix. PCR amplification was carried out with an initial cycle of heat activation at 98 degrees Celsius for 3 minutes seconds; 25 cycles of denaturation at 98 degrees Celsius for 30 seconds; 25 cycles of annealing at 62 degrees Celsius for 30 seconds; 25 cycles of extension at 72 degrees Celsius for 2 minutes; final extension at 72 degrees Celsius for 5 minutes; and finally holding the reaction at 4 degrees Celsius. Table 2 provides the PCR amplification program for enriching target nucleic acid regions, according to embodiments herein.

TABLE 2
PCR program for enriching target nucleic acid
regions, according to embodiments herein.

| Step | Temperature | Time | | Cycle |
|---|---|---|---|---|
| Heat activation | 98 | 3 | minutes | 1 |
| Denaturation | 98 | 30 | seconds | 25 |
| Annealing | 62 | 30 | seconds | 25 |
| Extension | 72 | 2 | minutes | 25 |
| Final Extension | 72 | 5 | minutes | 25 |

| | | | |
|---|---|---|---|
| Hold | 4 | Infinite | 1 |

### Example 3: DNA Repair and End-Prep Step

10 μl of normalized amplicons was dispensed in a fresh tube and processed for purification using purification beads. After purification, 10 μl of purified amplicons were mixed with 9 μl of the end repair buffer mix containing 1 μl of the end repair enzyme mix. The contents were gently mixed by pipetting and kept for incubation in a thermal cycler at 20 degrees Celsius for 15 minutes followed by heating at 65 degrees Celsius for 15 minutes.

### Example 4: Barcode Ligation

2 μl of provided barcode was added to 5 μl barcode mix. 5 μl of end prep reaction was added to the barcode mix tube. The reaction mixture was then incubated using a thermal cycler at 20 degrees Celsius for 25 minutes followed by heating at 65 degrees Celsius for 10 minutes.

### Example 5: Addition of Magnetic Beads and Quantification of Barcoded DNA

The contents of barcoded tubes were pooled into a 1.5 ml tube. The purification beads were added to the pooled tube at 2.5 times sample volume, followed by 5 minutes incubation at room temperature. The beads were kept on magnetic rack for 5 minutes. Once the beads move towards the magnet, the supernatant is removed. The tube is removed from the magnetic stand and the beads are resuspended by gentle pipetting in 500 μl of buffer SFB. The tube is again kept on magnetic stand for 5 minutes. Once a clear solution is obtained the supernatant is discarded and the pellet is washed with 80% ethanol. The tube was again kept on the magnetic rack until a clear and colorless supernatant was obtained. The supernatant was discarded and the tube was briefly spin and kept back the magnetic rack to allow the beads to separate. The residual ethanol was pipette off and the tube was allowed to dry partially for a few seconds. The tube was then removed from the magnetic rack and the pellet was resuspended in 32 μl Nuclease free water followed by 5-minutes incubation at room temperature. The beads were pelleted on a magnet until a clear and colorless elute was obtained. 30 μl of elute was removed and added in a fresh 1.5 ml tube.

### Example 6: Adapter Ligation, Clean-Up and Priming

15 μl solution adapter mix was added to the tube containing 5 μl of the enzyme. 30 μl elute was added to this tube and the contents were gently mixed. The 50 μl mixture was further incubated in a thermal cycler at 25 degrees Celsius for 20 mins. 20 μl purification beads were added to elute-adapter mixture and mixed gently by pipetting. The mixture was then incubated for 5 minutes at room temperature. The tube was further placed on a magnet to pellet the beads for 5 minutes until a clear and colorless elute was obtained. The supernatant was later on discarded using a pipette. The beads were then resuspended in 200 μl of buffer SFB and kept again on a magnetic stand for pelleting until a clear and colorless elute was obtained. The supernatant was later on discarded using a pipette. After repeating the previous step, the tube was kept for quick spinning and placed on a magnet. Residual supernatant was pipetted off. The tube was then removed from the magnetic rack and the pellet was resuspended in a 12 μl Elution buffer and kept on magnetic rack for 5-minute at room temperature. The beads were pelleted on a magnet until a clear and colorless elute was obtained. 10 μl of elute containing the DNA library was removed and added in a fresh 1.5 ml tube. The eluted sample was quantified using the Qubit™ fluorometer.

### Example 7: Sample Loading on SpotON Flow Cell

3 μl FLT was added to the 117 μl FLB tube and mixed by vortexing. A new MinION™ flow-cell was taken and placed onto the MinION™ by flipping open the lid and pushing one end of the flow-cell under the clip by pushing down gently. 800 μl of FLB (plus FLT) was loaded slowly into the flow cell via the inlet port so as to avoid introduction of any air bubble. After 5 minutes, the SpotON cover was gently lifted to open the SpotON port. 200 μl of additional FLB (plus FLT) was loaded into the flow cell via the inlet port to initiate a siphon at the SpotON port for allowing loading library dilution. The library dilution was prepared by adding 13.5 μl SQB, 11.5 μl LB and 5 μl final library. 29 μl of prepared library dilution was loaded into the flow cell via SpotON sample port in a dropwise fashion. SpotON sample port cover was gently replaced followed by closing the inlet port and MinION™ lid respectively. The MinKnowIM parameters were adjusted, and the run was started.

### Example 8: Computational Analysis of Generated Sequenced Data

Clinical Validation Preclinical testing was performed by testing 180 samples. This included 10 samples tested in various combinations and tested multiple times. This testing found a sensitivity and testing accuracy of >90%. Further, a pilot study was conducted using clinical isolates (n=70) and the results obtained from culture and using the said method were compared. This evaluation showed a high degree of concordance of 0.83 between said method and gold-standard culture tests. Further the analysis device/system was able to correctly identify species that were misidentified based on only culture profiles and similar biochemical characteristics.

### Example 9: Analytical Validation

The NGS data targeted for each sample was >500 reads at sequencing. At this depth, it is increasingly probable that the relevant reads matching the pathogen, if present, crosses the threshold (e.g., >100 reads for pure culture) after quality check. When gathering sequencing data using the ONT platform, a Q score of 8 was applied. The reads were checked for any human contamination. The raw reads obtained from sequencing were first checked for presence of chimeras and the read quality and those not passing both the criteria were filtered out (Q score=10). Finally, the reads are further inspected to fall between range of the expected fragment length (400-2500). This increased the confidence with which the presence of a pathogen or an ARG was confirmed. These filtered reads were then further evaluated to identify the closest match of the sample reads to the pathogens in the catalog. In order to identify a pathogen, a relevant match must be found. The curated database of the present invention includes known unique genomic signature sequences (e.g., 16 s regions) that can help accurately identify the pathogen or ARG. A pilot study compared the results using NGS with culture and found a concordance of 0.83.

The foregoing description of the specific embodiments will so fully reveal the general nature of the embodiments herein that others can, by applying current knowledge, readily modify and/or adapt for various applications such specific embodiments without departing from the generic concept, and, therefore, such adaptations and modifications should and are intended to be comprehended within the meaning and range of equivalents of the disclosed embodiments. It is to be understood that the phraseology or terminology employed herein is for the purpose of description and not of limitation. Therefore, while the embodiments herein have been described in terms of preferred embodiments, those skilled in the art will recognize that the embodiments herein can be practiced with modification within the spirit and scope of the embodiments as described herein.

1. A method for performing genomic analysis, wherein the method comprises:
pre-sequencing, by a pre-processing module (201), to obtain a polynucleotide library for sequencing, wherein the pre-sequencing is conducted by combining sequence specific primers;
performing sequencing, by the pre-processing module (201), by loading the polynucleotide library on a sequencing platform to obtain raw sequence data;
performing, by a genomic analysis module (202), basecalling and demultiplexing on the raw sequencing data to generate a first level of sequence data;
generating, by the genomic analysis module (202), the first quality score for the first level of the sequence data;
filtering, by the genomic analysis module (202), irrelevant reads of the sequence data from the first level of sequence data by removing irrelevant reads with quality scores that are below a pre-defined threshold;
classifying, by the genomic analysis module (202), potential reads of interest based on presence of primers and length of reads in the filtered data, which can be based on the presence of the sequence specific primers;
performing, by the genomic analysis module (202), annotation on the classified data;
interpreting, by the genomic analysis module (202), the annotated data based on sequencing level, quality thresholds, threshold of findings, and metadata; and
generating, by a report generation module (203), a report, based on the interpreted data and epidemiological context, wherein the report can designate one or more pathogen genus/species and Antibiotic Resistant Genes (ARG) identification.

2. The method, as claimed in claim 1, wherein said pre-sequencing comprises obtaining a sample;
extracting nucleic acid molecules from the sample;
enriching a plurality of target nucleic acid regions present in the sample by combining sequence specific primers to obtain targeted amplicons; and
processing the targeted amplicons to obtain a polynucleotide library for sequencing;
wherein, enriching is carried using an amplification technique.

3. The method, as claimed in claim 1, wherein said pre-sequencing comprises amplifying target nucleic acid regions present in a sample by introducing at least one primer sequence selected from a group consisting of SEQ ID NO. 1 to SEQ ID NO. 10.

4. The method, as claimed in claim 2, wherein the sample is selected from a group consisting of aseptic body fluids such as whole blood, plasma, serum, cerebrospinal fluid (CSF) or any fluid aspirate or tissue extracted from human subject, pus, bronchoalveolar lavage (BAL) sample, pleural fluid; non sterile body samples such as respiratory samples, sputum, urine, stool, mucus, saliva; tissue abscess, wound drainage and culture-based samples.

5. The method, as claimed in claim 1, wherein sequencing is performed by using a sequencing method selected from a group consisting of sequencing-by-synthesis (SBS), single-molecule Real Time sequencing (SMRT), nanopore sequencing, sequencing-by-ligation, sequencing-by-hybridization, solexa sequencing, Digital Gene Expression, Next generation sequencing, single molecule sequencing by synthesis (SMSS), massively-parallel sequencing, shotgun sequencing and Maxim-Gilbert sequencing.

6. The method as claimed in claim 2, wherein targeted amplicons are obtained by performing polymerase chain reaction amplification with an initial cycle of heat activation at 98 degrees Celsius for 3 minutes; 25 cycles of denaturation at 98 degrees Celsius for 30 seconds; 25 cycles of annealing at 62 degrees Celsius for 30 seconds; 25 cycles of extension at 72 degrees Celsius for 2 minutes; final extension at 72 degrees Celsius for 5 minutes; and finally holding the reaction at 4 degrees Celsius.

7. The method, as claimed in claim 1, wherein the method comprises removing the irrelevant reads based on basis length, reads with low quality, chimeric sequences, host DNA, high-N-content reads and repeated sequences, DNA over-amplification, reads corresponding to barcodes, adapters ligated to the DNA fragment for the sequencing purpose, and primers used for amplification.

8. The method, as claimed in claim 1, wherein the method comprises performing annotation on the filtered data using at least one of sequence alignment, sequence assembly, contiguous sequence similarity, consensus, sequence similarity, GC content, average nucleotide identity, maximum parsimony, maximum likelihood, relative distance, cladistic single nucleotide polymorphisms, and time to most recent common ancestor.

9. The method, as claimed in claim 7, wherein the method comprises performing pathogen annotation using a custom database, wherein performing pathogen annotation comprises size matching, matching of sequences to target pathogen regions delineating information such as pathogen genus, and species or antibiotic resistance genes.

10. The method, as claimed in claim 7, wherein the method comprises performing ARG annotation using a custom database.

11. The method, as claimed in claim 1, wherein the metadata comprises patient metadata; clinical criteria; treatment related criteria; laboratory criteria and biomarker information.

12. The method, as claimed in claim 1, wherein the report includes a score, wherein the score can indicate the probability of infection in the patient, wherein the score is based on one or more pre-decided criteria, decision matrix, and Machine Learning (ML) models.

13. A system for performing genomic analysis, wherein the system comprises:
a pre-processing module (201);
a genomic analysis module (202); and
a report generation module (203);
wherein the pre-processing module (201) is configured for:
performing pre-sequencing to obtain a polynucleotide library for sequencing, wherein the pre-sequencing is conducted by combining sequence specific primers; and
performing sequencing by loading the polynucleotide library on a sequencing platform to obtain raw sequence data;
wherein the genomic analysis module (202) is configured for:
performing basecalling and demultiplexing on the raw sequencing data to generate a first level of sequence data;
generating, the first quality score for the first level of the sequence data;
filtering, irrelevant reads of the sequence data from the first level of sequence data by removing irrelevant reads with quality scores that are below a pre-defined threshold;
classifying, potential reads of interest based on presence of primers and length of reads in the filtered data, which can be based on the presence of the sequence specific primers;
performing, annotation on the classified data; and
interpreting the annotated data based on sequencing level, quality thresholds, threshold of findings, and metadata; and
wherein the genomic analysis module (202) is configured for:
generating a report, based on the interpreted data and epidemiological context, wherein the report can designate one or more pathogen genus/species and Antibiotic Resistant Genes (ARG) identification.

14. The system, as claimed in claim 13, wherein the pre-processing module (201) is configured for performing said pre-sequencing by:
obtaining a sample;
extracting nucleic acid molecules from the sample;
enriching a plurality of target nucleic acid regions present in the sample by combining sequence specific primers to obtain targeted amplicons; and
processing the targeted amplicons to obtain a polynucleotide library for sequencing;
wherein, enriching is carried using an amplification technique.

15. The system, as claimed in claim 13, wherein said pre-sequencing comprises amplifying target nucleic acid regions present in a sample by introducing at least one primer sequence selected from a group consisting of SEQ ID NO. 1 to SEQ ID NO. 10.

16. The system, as claimed in claim 14, wherein the sample is selected from a group consisting of aseptic body fluids such as whole blood, plasma, serum, cerebrospinal fluid (CSF) or any fluid aspirate or tissue extracted from human subject, pus, bronchoalveolar lavage (BAL) sample, pleural fluid; nonsterile body samples such as respiratory samples, sputum, urine, stool, mucus, saliva; tissue abscess, wound drainage and culture-based samples.

17. The system, as claimed in claim 13, wherein sequencing is performed by using a sequencing method selected from a group consisting of sequencing-by-synthesis (SBS), single-molecule Real Time sequencing (SMRT), nanopore sequencing, sequencing-by-ligation, sequencing-by-hybridization, solexa sequencing, Digital Gene Expression, Next generation sequencing, single molecule sequencing by synthesis (SMSS), massively parallel sequencing, shotgun sequencing and Maxim-Gilbert sequencing.

18. The system, as claimed in claim 14, wherein targeted amplicons are obtained by performing polymerase chain reaction amplification technique with an initial cycle of heat activation at 98 degrees Celsius for 3 minutes; 25 cycles of denaturation at 98 degrees Celsius for 30 seconds; 25 cycles of annealing at 62 degrees Celsius for 30 seconds; 25 cycles of extension at 72 degrees Celsius for 2 minutes; final extension at 72 degrees Celsius for 5 minutes; and finally holding the reaction at 4 degrees Celsius.

19. The system, as claimed in claim 13, wherein the method comprises removing the irrelevant reads based on basis length, reads with low quality, chimeric sequences, host DNA, high-N-content reads and repeated sequences, DNA over-amplification, reads corresponding to barcodes, adapters ligated to the DNA fragment for the sequencing purpose, and primers used for amplification.

20. The system, as claimed in claim 13, wherein the method comprises performing annotation on the filtered data using at least one of alignment, and consensus.

21. The system, as claimed in claim 20, wherein the method comprises performing pathogen annotation using a custom database, wherein performing pathogen annotation comprises size matching, matching of sequences to target pathogen regions delineating information such as pathogen genus, and species or antibiotic resistance genes.

22. The system, as claimed in claim 20, wherein the method comprises performing ARG annotation using a custom database.

23. The system, as claimed in claim 13, wherein the metadata comprises patient metadata; clinical criteria; treatment related criteria; laboratory criteria and biomarker information.

24. The system, as claimed in claim 13, wherein the report includes a score, wherein the score can indicate the probability of infection in the patient, wherein the score is based on one or more pre-decided criteria, decision matrix, and Machine Learning (ML) models.

25. A kit for performing genomic analysis for detection and identification of pathogens and antibiotic resistance genes, the kit comprising:
at least one vial of sequence specific primer mix; and
an instruction manual.


SEQUENCE LISTING (10 sequences)

SEQ ID NO: 1 - DNA, 22 bp, Synthetic construct
  source 1..22: mol_type = other DNA
catttccgtgtcgcccttattc

SEQ ID NO: 2 - DNA, 22 bp, Synthetic construct
  source 1..22: mol_type = other DNA
cgttcatccatagttgcctgac

SEQ ID NO: 3 - DNA, 21 bp, Synthetic construct
  source 1..21: mol_type = other DNA
agccgcttgagcaaattaaac

SEQ ID NO: 4 - DNA, 21 bp, Synthetic construct
  source 1..21: mol_type = other DNA
atcccgcagataaatcaccac

SEQ ID NO: 5 - DNA, 22 bp, Synthetic construct
  source 1..22: mol_type = other DNA
ggcaccagattcaactttcaag

SEQ ID NO: 6 - DNA, 22 bp, Synthetic construct
  source 1..22: mol_type = other DNA
gaccccaagtttcctgtaagtg

SEQ ID NO: 7 - DNA, 22 bp, Synthetic construct
  source 1..22: mol_type = other DNA
cttggtcatttagaggaagtaa

SEQ ID NO: 8 - DNA, 20 bp, Synthetic construct
  source 1..20: mol_type = other DNA
tcctccgcttattgatatgc

SEQ ID NO: 9 - DNA, 20 bp, Synthetic construct
  source 1..20: mol_type = other DNA
agagtttgatcctggctcag

SEQ ID NO: 10 - DNA, 22 bp, Synthetic construct
  source 1..22: mol_type = other DNA
tacggytaccttgttacgactt